(19) United States
(12) Patent Application Publication
(10) Pub. No.: US 2018/0055134 A1
(43) Pub. Date: Mar. 1, 2018

(54) Multifunctional and Intelligent Industrial Helment

(71) Applicant: Zhaoqing Bohan Sports Products Limited, Zhaoqing City (CN)

(72) Inventor: Ming Zhang, Fo Shan City (CN)

(21) Appl. No.: 15/499,937

(22) Filed: Apr. 28, 2017

(30) Foreign Application Priority Data
Aug. 23, 2016 (CN) 201620917669.1

(51) Int. Cl. A42B 3/30; H04N 5/225; H04R 1/10; G01N 33/00; A61B 5/00; A42B 3/04; A42B 3/10

(52) U.S. Cl. CPC A42B 3/303; H04N 5/2257; H04N 5/2253; H04N 5/2252; H04R 1/1066; H04R 2201/107; A61B 5/6803; A42B 3/042; A42B 3/046; A42B 3/10; G01N 33/0009

(57) ABSTRACT

A multifunctional and intelligent industrial helmet is provided, comprising a helmet body and a lining layer provided on the helmet body, wherein a multifunctional control system is further provided within the helmet body; the multifunctional control system comprises a main control module, and a harmful gas detection module, a talkback module, a camera module, a power supply module, a storage module and a network module, all of which are electrically connected to the main control module; and, during the operation, the camera module records videos or takes pictures and stores the videos or pictures in the storage module, or transmits the videos or pictures to a background server in real time via the network module.

### CROSS REFERENCE TO RELATED APPLICATION

This application takes priority from and claims the benefit of Chinese Patent Application No. 201620917669.1, filed 23 Aug. 2016, the contents of which are hereby incorporated by reference in their entirety.

### TECHNICAL FIELD

The present utility model relates a helmet and in particular to a multifunctional and intelligent industrial helmet.

### BACKGROUND OF THE PRESENT INVENTION

Unforeseen circumstances in the fire rescue scenes and in the industrial production processes will threaten the personal safety of firefighters and numerous casualty accidents of firefighters happen all over the world every year. Therefore, it is very important for on-site operations to enhance the personal protection of firefighters. During on-site operations, if information about the on-site environment and information about firefighters can be acquired in real time, the firefighters can timely make an effective judgment on potential hazards and thus avoid casualty accidents. A fire commander can timely make an effective decision according to the scene information, so as to optimally dispatch on-site operations and better command firefighters. Under the premise of ensuring the personal safety of firefighters, both lives and property are secured as far as possible in the operating site.

Conventional helmets for firefighters and helmets for industrial workers are still stuck at passive protection, and lack of informatization and intelligentization functions for information perception and wireless transmission. In the era of rapid development of internet and big data, the intelligent fire-fighting equipment has become a development trend of equipment in the fire protection industry, and fire-fighting on-site operations have a strong demand for intelligentized information perception and command dispatching. The innovation and upgrading of the fire-fighting equipment facilitates the evolution from functional equipment to intelligent equipment.

### SUMMARY OF THE PRESENT INVENTION

An objective of the present utility model is to provide a multifunctional and intelligent industrial helmet which is simple in structure, easy to use and high in informatization level, in order to overcome the disadvantages in the prior art.

The objective of the present utility model is realized in the following ways. A multifunctional and intelligent industrial helmet is provided, including a helmet body and a lining layer provided on the helmet body, wherein a multifunctional control system is further provided within the helmet body; the multifunctional control system includes a main control module, and a harmful gas detection module, a talkback module, a camera module, a power supply module, a storage module and a network module, all of which are electrically connected to the main control module; and, during the operation, the camera module records videos or takes pictures and stores the videos or pictures in the storage module, or transmits the videos or pictures to a background server in real time via the network module.

The multifunctional control system further includes an SOS module; and, when a user presses down an SOS button, the SOS module is activated and transmits an SOS signal via the talkback module and the network module.

The talkback module includes a microphone module and an earphone module both provided on the helmet body, and the earphone module is a bone-conduction earphone or an earplug-type earphone.

The network module is a composite transmission network based on WiFi, Bluetooth and 2G/3G/4G communication, and automatically switches between connection modes according to various scenes.

The harmful gas detection module is provided with a connection receptacle on the helmet body, and can be separately or simultaneously connected to multiple gas sensors according to environment needs.

The storage module is an SD or TF storage card which is detachably mounted on the helmet body.

The main control module is further electrically connected to a vital sign detection module for detecting vital signs of a wearer in real time.

The camera module comprises a camera body and a housing covered on the surface of the camera body, and the housing is connected to the helmet body to fix the camera body.

The present utility model has the following beneficial effects: first, with a simple structure and a low production cost, the helmet is highly competitive; second, the helmet has a real-time talkback function, so it is convenient for multi-party talkback, real-time conversion and instruction to a foreground wearer from the background, and the like; third, with the function of harmful gas detection and alarming and the reservation of multiple sensor interfaces, the helmet is suitable for the harmful gas monitoring in different occasions according to different requirements of different scenes, so that the occurrence of hazards is prevented; and, four, since multiple network communication modes are provided and automatic switching between those network communication modes according to on-site situations can be realized, it is ensured that live videos can be passed-back by the video module.

### BRIEF DESCRIPTION OF THE DRAWINGS

FIG. 1 is an overall assembly effect diagram of the present utility model;

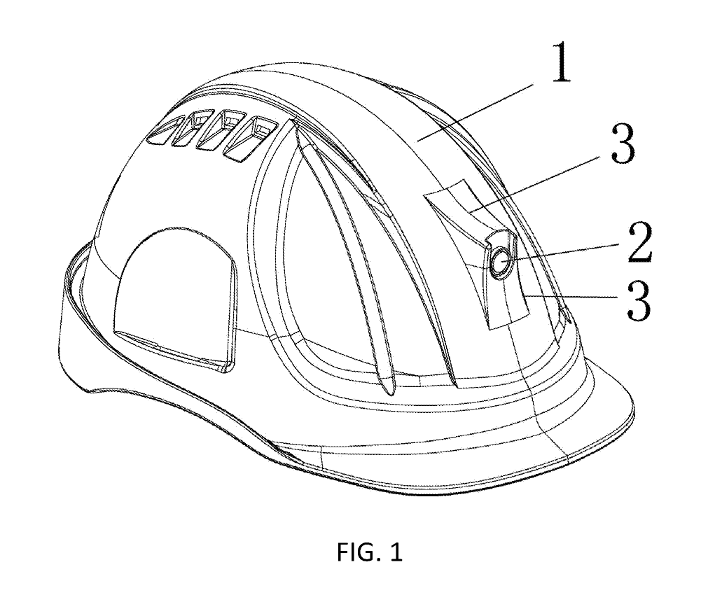

FIG. 2 is an assembly effect diagram of a camera according to the present utility model; and

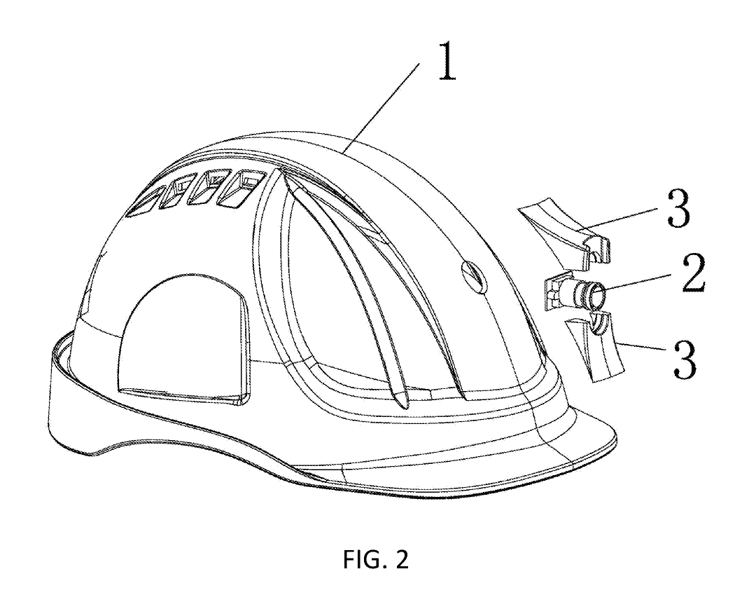

FIG. 3 is an electrical principle diagram of the present utility model.

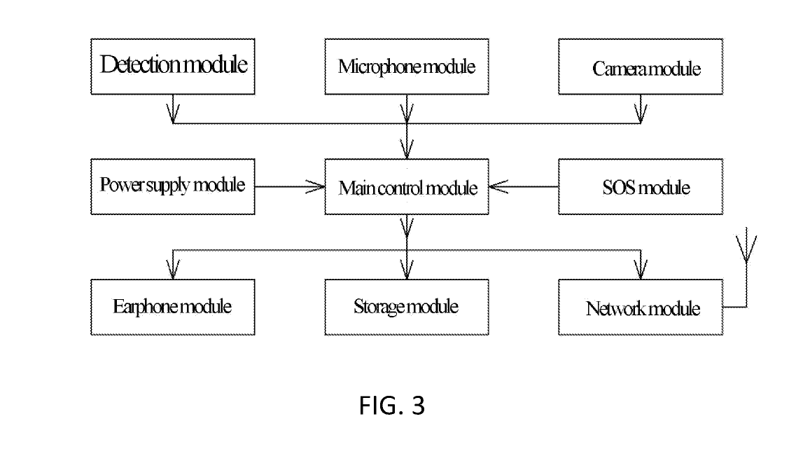

### DETAILED DESCRIPTION OF THE PRESENT INVENTION

The present utility model will be further specifically described below with reference to the accompanying drawings. A multifunctional and intelligent industrial helmet is provided, including a helmet body 1 and a lining layer provided on the helmet body 1, wherein a multifunctional control system is further provided within the helmet body 1; the multifunctional control system includes a main control module, and a harmful gas detection module, a talkback module, a camera module, a power supply module, a storage module and a network module, all of which are electrically connected to the main control module; and, during the operation, the camera module records videos or takes pictures and stores the videos or pictures in the storage module, or transmits the videos or pictures to a background server in real time via the network module.

The multifunctional control system further includes an SOS module; and, when a user presses down an SOS button, the SOS module is activated and transmits an SOS signal via the talkback module and the network module.

The talkback module includes a microphone module and an earphone module both provided on the helmet body, and the earphone module is a bone-conduction earphone or an earplug-type earphone.

The network module is a composite transmission network based on WiFi, Bluetooth and 2G/3G/4G communication, and automatically switches between connection modes according to various scenes.

The harmful gas detection module is provided with a connection receptacle on the helmet body, and can be separately or simultaneously connected to multiple gas sensors according to environment needs.

The storage module is an SD or TF storage card which is detachably mounted on the helmet body.

The main control module is further electrically connected to a vital sign detection module for detecting vital signs of a wearer in real time.

The camera module includes a camera body 2 and a housing 3 covered on the surface of the camera body 2, and the housing 3 is connected to the helmet body 1 to fix the camera body 2.

The operating principle is as follows: after a user wears the helmet, the main control module activates the network module, the camera module, the detection module and the talkback module, wherein the network module is a composite transmission network adopting WiFi, Bluetooth and 2G/3G/4G communication, and the user may switch between network connection modes according to the practical situation of a scene so as to adapt to various scenes, so that the main control module can be connected to a background system in real time.

Wherein, the talkback module includes a microphone module and an earphone module. In cooperation with the camera module, the control system may transmit the shot audios and videos to the background via the network module, and store the audios and videos in the local storage module. The storage module may be an SD, a TF storage card or other storage media, and will not be limited specifically herein. Wherein, the talkback module may realize multi-party talkback, real-time conversion and instruction to a foreground wearer from the background, and the like. To improve the comfort for wearing, the earphone module herein may be a conventional earplug earphone, or may be a more advanced bone-conduction earphone. Multiple configurations may be provided for the earphone module during its production for users to choose.

Further, to be used in dangerous industries such as outdoor adventure, geological exploration, fire-fighting operations and chemical engineering, the helmet provided by the present utility model is further provided with a harmful gas detection module. The harmful gas detection module is provided with a connection receptacle on the helmet body, and can be separately or simultaneously connected to multiple gas sensors according to environment needs. Once the system detects a harmful gas via a sensor, the system will timely send alarm information to remind the user. If necessary, the system transmits alarm information to the background via the network module, so that the safety of the user is enhanced.

Further, the main control module is further provided with an SOS module. When the situation is out of control and the background fails to find the user timely, the user may press down an SOS button, and the main control module may judge whether a danger happens or not via its own sensor. If necessary, location information of the user is determined by location information (LBS), and then transmitted to the background for rescuing of the background.

Further, the main control module is further electrically connected to a vital sign detection module which may detect vital signs such as blood pressure, heartbeat, blood oxygen concentration and the like of a wearer in real time. Thus, it is convenient for the background to learn about the physical state of the user. The helmet is particularly suitable for firefighters, users for outdoor adventure and the like.

Compared with the conventional technologies, the helmet provided herein has various advanced detection functions and real-time communication systems, and can detect the indexes of the environment safety and vital signs of the user. If necessary, the helmet can interact with a background management and monitoring system via a video and voice pass-back function, so that it is convenient for implementing rescue and production plans and the like. Hence, the helmet can be widely popularized.

The basic principle and main features of the present utility model and the advantages of the present utility model have been shown and described above. It should be understood by those skilled in the art that the present utility model is not limited to the embodiments described above, and those embodiments and descriptions are merely used for describing the principle of the present utility model; and, various variations and improvements may be made to the present utility model without departing from the spirit and scope of the present utility model, and these variations and improvements shall fall into the protection scope of the present utility model.

1. A multifunctional and intelligent industrial helmet, comprising a helmet body (1) and a lining layer provided on the helmet body (1), characterized in that a multifunctional control system is further provided within the helmet body (1); the multifunctional control system comprises a main control module, and a harmful gas detection module, a talkback module, a camera module, a power supply module, a storage module and a network module, all of which are electrically connected to the main control module; and, during the operation, the camera module records videos or takes pictures and stores the videos or pictures in the storage module, or transmits the videos or pictures to a background server in real time via the network module.

2. The multifunctional and intelligent industrial helmet according to claim 1, characterized in that the multifunctional control system further comprises an SOS module; and, when a user presses down an SOS button, the SOS module is activated and transmits an SOS signal via the talkback module and the network module.

3. The multifunctional and intelligent industrial helmet according to claim 1, characterized in that the talkback module comprises a microphone module and an earphone module both provided on the helmet body, and the earphone module is a bone-conduction earphone or an earplug-type earphone.

4. The multifunctional and intelligent industrial helmet according to claim 1, characterized in that the network module is a composite transmission network based on WiFi, Bluetooth and 2G/3G/4G communication, and automatically switches between connection modes according to various scenes.

5. The multifunctional and intelligent industrial helmet according to claim 1, characterized in that the harmful gas detection module is provided with a connection receptacle on the helmet body, and can be separately or simultaneously connected to multiple gas sensors according to environment needs.

6. The multifunctional and intelligent industrial helmet according to claim 1, characterized in that the storage module is an SD or TF storage card which is detachably mounted on the helmet body.

7. The multifunctional and intelligent industrial helmet according to claim 1, characterized in that the main control module is further electrically connected to a vital sign detection module for detecting vital signs of a wearer in real time.

8. The multifunctional and intelligent industrial helmet according to claim 1, characterized in that the camera module comprises a camera body (2) and a housing (3) covered on the surface of the camera body (2), and the housing (3) is connected to the helmet body (1) to fix the camera body (2).
